(19) United States
(12) Patent Application Publication
(10) Pub. No.: US 2021/0266279 A1
(43) Pub. Date: Aug. 26, 2021

(54) Audio-First Health Conversations Platform

(71) Applicant: Anecure Inc., San Francisco, CA (US)

(72) Inventors: Sudha Kanakambal Varadarajan, San Francisco, CA (US); Arish Ali, San Francisco, CA (US); Daniel Latham Hastings, Portland, OR (US)

(21) Appl. No.: 17/180,657

(22) Filed: Feb. 19, 2021

Related U.S. Application Data
(60) Provisional application No. 62/979,339, filed on Feb. 20, 2020.

(51) Int. Cl. H04L 12/58; G06F 3/16; G06Q 50/00; G16H 80/00

(52) U.S. Cl. CPC H04L 51/10; G06F 3/16; G16H 80/00; G06Q 50/01; H04L 51/32

(57) ABSTRACT

One aspect of the invention is an audio-first social platform focused on health, in the form of a website, an application or other digital form, where users and businesses come together to share information on ailments, treatments, cures, therapies and other health care related activities. In this platform users will be able to share their experiences with other users, in a peer to peer manner. The primary mode of communication is audio, with visuals to augment audio as the need may be.

### CROSS-REFERENCE TO RELATED APPLICATION

This application claims priority under 35 U.S.C. § 119(e) to U.S. Provisional Patent Application Ser. No. 62/979,339, “Audio-First Health Conversations Platform,” filed Feb. 20, 2020. The subject matter of all of the foregoing is incorporated herein by reference in their entirety.

### BACKGROUND

### 1. Technical Field

This disclosure relates generally to health care platforms.

### 2. Description of Related Art

When it comes to health, we need a medium that can best express our emotions such as anxiety, pain and joy when it comes to describing health issues. Most health-related websites and platforms use text as the primary mode of communication. You share your health experience by writing about it in a text form field or by sending via email or some form of text messaging. While some platforms may allow submitting additional media such as photos and videos, the subsequent discussion and replies tend to happen via text. These platforms are not able to capture all the nuances of emotions and authenticity in a text based interaction.

### BRIEF DESCRIPTION OF THE DRAWINGS

Embodiments of the disclosure have other advantages and features which will be more readily apparent from the following detailed description and the appended claims, when taken in conjunction with the examples in the accompanying drawings, in which:

FIG. 1 is a block diagram of a central system according to the present invention.

FIG. 2 is a block diagram of a user-side module according to the present invention.

FIG. 3 is a screen shot of an app according to the present invention.

### DETAILED DESCRIPTION OF THE PREFERRED EMBODIMENTS

The figures and the following description relate to preferred embodiments by way of illustration only. It should be noted that from the following discussion, alternative embodiments of the structures and methods disclosed herein will be readily recognized as viable alternatives that may be employed without departing from the principles of what is claimed.

When it comes to health, we need a medium that can best express our emotions such as anxiety, pain and joy when it comes to describing health issues. As humans, our social interactions have traditionally involved using the spoken voice to interface with others. We create a digital media platform to bring together users discussing health and health concerns and treatments, with audio as the primary means of interfacing with others when it comes to discussing health issues and ailments. A digital platform that uses audio as the primary type of content to communicate between users and is focused on health and conversations about health.

One aspect relates to a digital media platform for sharing audio content between users about health. Audio, user generated and other, is the primary medium of content that is posted by users and shared amongst one another.

Social media platforms such as Facebook, Twitter, Instagram, Snapchat, YouTube and Whatsapp exist today to enable social interactions between users. The primary method of interaction is usually text, a photo or a video. They are not an audio-first platform where the type of content shared by an individual is first audio which is then shared and interacted with. Moreover, they are not platforms that are focused on health and health issues, so they allow posting of all forms of content. A health-based platform may structure site data and data structures around ailments and cures and may group discussions based on diseases and treatments, which is not the focus of the existing social interaction platforms.

The use of audio is unique. It preserves anonymity without sacrificing authenticity. Video does not work because it reveals the identity of the person speaking, and text-based platforms do not capture the emotional impact of the story. Text based platforms are also more susceptible to fake content as the effort to make a text only post is very low. In addition, historically, people have communicated about health issues through conversations—whether it is conversations with their doctor, other health providers or simply talking with friends and family over dinner. So audio is the natural medium for health stories and anecdotes, which is what this platform captures.

Human beings from around the world and businesses from around the world can use this social platform and lend their voices to discuss ideas and share thoughts with one another regarding health, health concerns, treatments and cures. This would be similar to how humans have traditionally interacted with one another and take this interaction into a digital space to allow this interaction in a synchronous and asynchronous manner in a digital medium.

Health discussions require paying close attention to the issues being discussed, the symptoms, diagnosis, treatments and therapies. Voice based communication of thoughts and ideas are more natural and authentic. The listener is engaged with what is being said—listening to the idea and emotion in the communication—and is able to engage with the emotions behind the message, which text may not fully convey. This can bring about a significant change in social communication, especially when exchanging health conversations in an asynchronous manner, and starting dialogue across communities.

One aspect of the invention is an audio-first social platform focused on health, in the form of a website, an application or other digital form, where users and businesses come together to share information on ailments, treatments, cures, therapies and other health care related activities.

In this platform users will be able to share their experiences with other users, in a peer to peer manner. The primary mode of communication is audio, with visuals to augment audio as the need may be.

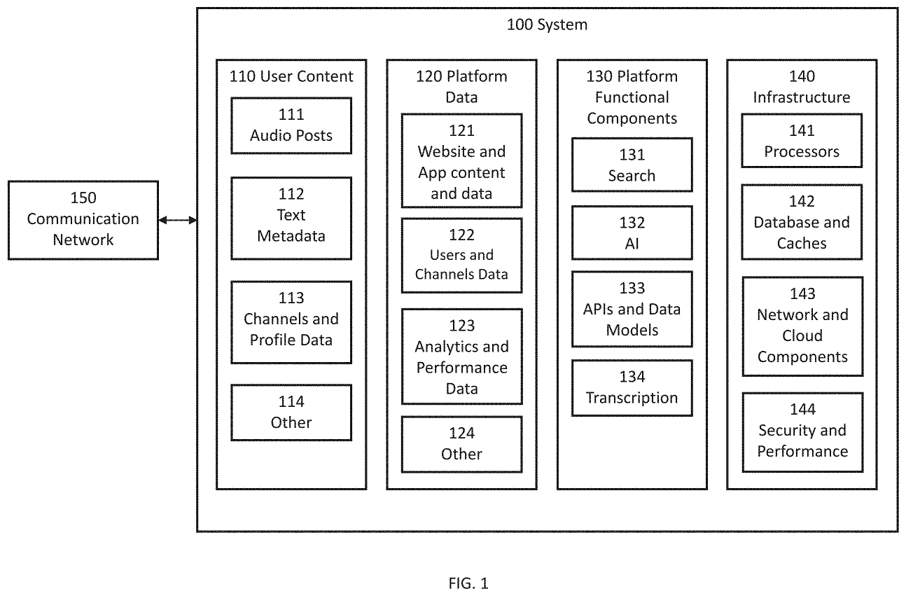

FIG. 1 is a block diagram of a central system 100 according to the present invention. The system 100 may include the following:
110: User Content: All the user data, including the following:
111: Audio Posts: The actual voice recordings of the user.
112: Text Metadata: The metadata about the audio, which may include title, description, tags, links and other information.
113: Channels and Profile Data: Information about the different channels the user content is posted in and also the personal profile of the user.
114: Other: Other user related data required for the system, including but not limited to geographical and language data.
120: Platform Data: All the platform data, including the following;
121: Website and App content and data: The data required to render the website and app for the full experience to the user.
122: Users and Channels Data: Data related to all the users and their channels.
123: Analytics and Performance Data: Data about system usage and analytics and trends, and also data used to improve performance.
124: Other: Other platform data.
130: Platform Functional Components: The components embodying the functionality of the application, including:
131: Search: The functionality to allow a user to find the content they want to hear.

For example, if they are looking for a story related to a particular disease like cancer.
132: AI: The Artificial Intelligence system.
133: APIs and Data Models: The Application Programming Interface (“API”) that allows access to system functionality to other components of the system, as well as to remote web or mobile application (for example, module 200).
134: Transcription: For converting audio to text data.
140: Infrastructure: The hardware and networking components required to host and make the system function, including:
141: Processors: The computational processors (CPUs) where the system executes.
142: Database and Caches: For persistent and transient storage of all the data in the system.
143: Network and Cloud Components: The components that provide networking functionality such as load balancing, edge caching, firewalls etc.
144: Security and Performance: For monitoring the system against hacks and intrusion, and also for optimizing the performance of the platform.
150: Communication Network: This is the internet, used by module 200 to communicate and interact with module 100.

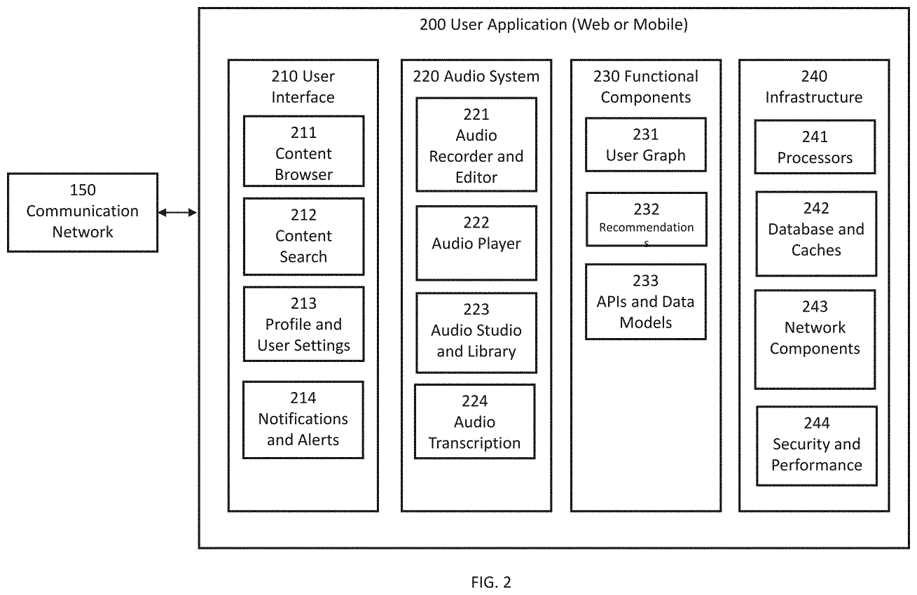

FIG. 2 is a schematic of the user-side module 200. The user application 200 is the front end application that the user interacts with. It can be web based or a mobile application (for example, an iOS app or an Android app). It may include the following:
210: User Interface: The graphic interface used by the user to interact with application and the system;
211: Content Browser: Allows the user to discover content from other users.
212: Content Search: Allows the user to search for content or other users or channels or other data from the system.
213: Profile and User Settings: Information about the personal profile of the user, user preference and profile data.
214: Notifications and Alerts: Notifies the user of new content and activity in the system.
220: Audio System: All the audio specific features, including the following:
221: Audio Recorder and Editor: Allows the user to record an audio post, edit and re-record it.
222: Audio Player: Allows the user to listen to the audio content in the application.
223: Audio Studio and Library: Allows the user to modify the audio recording by adding additional audio components from the library and also to modify the audio, for example by automatically reducing noise.
224: Audio Transcription: Converts the audio to text.
230: Functional Components: The components embodying the functionality of the application, including:
231: User Graph: Includes the functionality of connecting various users and user data such as the channels and the stories they have posted.
232: Recommendations: Shows stories recommended for the user based on stories the user has already listened, for example.
233: APIs and Data Models: The Application Programming Interface (“API”) that allows access to system functionality to other components of the system (module 100).
240: Infrastructure: The hardware and networking components required to host and make the system function:
241: Processors: The computational processors (CPUs) where the system executes. For a mobile application, this will be in the mobile phone.
242: Database and Caches: For persistent and transient storage of all the data in the web application or the mobile device.
243: Network Components: The components that provide access to resources over the network
244: Security and Performance: For monitoring the system against hacks and intrusion, and also for optimizing the performance of the platform.

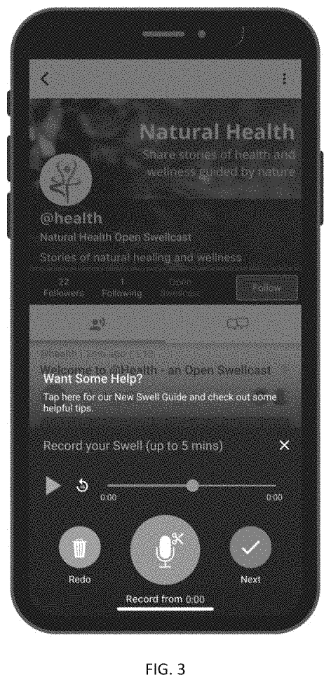

FIG. 3 is a screenshot of the Apple iOS version of the mobile application showing the audio recorder functionality (module 221 of user application 200).

A user comes into the sites and wishes to post a health related conversational message (HCM). As he goes to create his message, he is presented with an audio recorder interface. He can speak what he wishes to convey and it is recorded. He may optionally save and resume his recording at a later point. He may also record over some elements of his recording, should he wish to alter some of his content. He then posts his recording and it is made available to all other users. Alternatively, a user may choose to upload an audio file or stream that he recorded using an alternate device. He then indicates what type of message it is, such as a message about a health cure or control or therapy, who it is about (himself, his or her child, etc), the ailment being discussed, and some personal information such as the age, gender and location of the person with the issue. Some of these elements may automatically get filled in using AI and language analysis of the user's message.

The user may be provided a digital audio studio which he can use to edit the HCM. Some examples of such edits are:
Intersperse sounds and music from a library of available sounds and music in the studio and in the user's device or computer into the audio message by overlaying tracks to play in parallel, in sequence, fade in and out and such.
Morph the tracks to stretch, shorten or otherwise alter the voices to induce effects that can be humorous, dramatic or other.
Identify and tag different users whose voices are in the audio. This may happen automatically as well as using AI and the user can edit the AI's tagging, should he wish to.

Once a user has created his HCM, he goes ahead and posts it and at that time can choose to limit the audience for his message—to be heard by all users, to be heard only by his followers, to be heard by a specific individual(s), or to be heard by a specific group(s) of individuals.

A business entity may establish its presence in this platform and a spokesperson of the business may create an HCM on behalf of the business in a manner similar to how an individual does it. He then posts the message on behalf of the business and can make it available to all users of the platform or just the followers of the business or a particular group(s) of users.

A user may also create one or more “channels” to post one or more HCMs in each channel. A channel will allow the user to group together HCMs on a particular topic or having some common theme or it can be any arbitrary collection of HCMs recorded and posted by the user. The user can also go and interview or solicit recordings from other users to create HCMs for posting in these channels. Other users can subscribe to or otherwise interact with a particular channel or specific HCMs in a channel.

When an HCM is submitted, it may be automatically transcribed and converted to text using an AI and/or human enabled system. The transcribed text is displayed next to the audio. When the HCM plays back, the system may choose to highlight or otherwise bring to the front that portion of the transcribed text that is currently being spoken. The transcription may also be automatically by an AI and/or with the help of humans translated into multiple other languages.

A submitted HCM may also be translated to other languages in audio either automatically using an AI or human-enabled and the audio message may be made available in audio-translated versions.

Health related data elements may be automatically extracted from the HCM using an AI and schematic fields of a data model may be automatically populated. For example, if the HCM was one about a health issue, schematic elements such as the ailment, the disease, the treatment, and such may be automatically learnt from the audio and data elements may be populated that complement the audio portion of the message. Some of these auto-learnt and extracted data elements may be presented to viewers alongside the audio message, while others may be used internally for purposes of business intelligence and learning.

When a person views someone else's HCM and wishes to applaud or reject it, he can do so using an audio based applause or jeer. More generally, while listening to an HCM, a user can provide a range of emotional responses at various points (applause, laugh, cry, feel sad, feel happy etc.), each of which will be easily enabled by the user interface. These emotional responses are captured along with the time of the response (at what time in the audio recording did the user give a particular emotional response).

Subsequently, when someone else is listening to the same HCM, they will be able to view the emotional responses from other users at the corresponding times in the playback. So, for example, if many users gave a “laugh” response at 30 seconds into the audio timeline, then the user interface will indicate those multiple laugh events when the user reaches that 30 second mark in the listening experience. These laugh events can be shown visually or can form their own audio “overlay” audio soundtrack (similar to a laugh track in a TV show) which can be turned on or off. This provides a unique “co-listening” experience where the more users listen and respond to a particular HCM, the richer the listening experience becomes for each subsequent listener.

The addition of user emotional responses at various points in the story soundtrack provides us with a unique set of additional data that can be used in conjunction with the original story in our AI data models to come up with insights regarding the quality and effectiveness of various health solutions and approaches captured in the HCMs in the platform. This use of an ever growing (with each listener) body of emotional response data to augment existing health data in a story is unique and not something that has been done in existing applications of AI.

When someone wishes to comment on someone else's HCM, he can do so by recording an audio comment. He is presented with a similar recording option as when creating a new message and has the studio to help him edit his comment and add bells and whistles to it. He can then post his audio comment, which may be transcribed and translated upon submission.

Various aspects of this disclosure include: 1. a digital space (application, website or other) for human and business communication on health care that is audio-first and may additionally have any of the following:

2. Provides an audio recorder to create audio based health conversation messages (HCM) to post to an individual, a group of users, followers, or everyone. Optionally, wherein said recorder allows the user to save and resume his audio recording, before posting. Optionally, wherein said recorder allows the user to edit his recording in a particular area or timeslot.

3. Permits uploading an audio file or stream HCM to post to an individual, a group of users, followers, or everyone. Optionally, wherein said audio file or stream was generated or obtained by user in an alternate system.

4. May provide an audio studio to edit the HCM. Optionally, wherein the audio studio may allow editing of the HCM with audio effects and sound effects. Optionally, wherein the audio studio may allow programmatically and/or manually identifying and tagging users whose voices are in the HCM.

5. May provide transcription of the HCM. Optionally, wherein the system may programmatically and/or with the help of human transcribe the audio to text. Optionally, wherein the system may programmatically and/or with the help of human translate the transcription of the audio to other languages. Optionally, wherein the system may programmatically highlight the portion of the transcribed text that is being spoken when the audio is played back.

6. May provide translation of the HCM. Optionally, wherein the system may programmatically and/or with the help of human translate the audio to audio in other languages.

7. May provide programmatic extraction of data from the HCM. Optionally, wherein the system may programmatically extract data elements from the HCM by semantic and/or natural language understanding of what is spoken. Optionally, wherein the system may display the extracted data alongside the HCM and/or use it for internal business intelligence.

8. May provide audio based applause or rejection of posts. Optionally, wherein the system presents the listener of an audio post the ability to applaud or reject a message using audio based applause or jeer.

9. May provide audio based comments to posts. Optionally, wherein the system presents the listener of an audio post the ability to comment on the message using audio.

10. May provide audio and/or visual emotional reactions to HCM, Optionally, wherein a system presents the listener of the audio post ability to select from and provide a range of emotions (laugh, cry, sad, happy etc.) at various points of time while listening.

11. May provide a “co-listening” experience where other listener's emotional reactions as captured in (i) above are presented to subsequent listeners at the same point of time through visual or audio means as an overlay visual and/or overlay soundtrack.

12. May provide the ability to create “channels” to group multiple HCMs. Optionally, wherein other users can interact with and/or subscribe to a channel.

13. May generate insights using AI data models and algorithms that take into account the raw audio story provided in the HCM in conjunction with the emotional response timeline provided by various consumers of the HCM in the platform.

Although the detailed description contains many specifics, these should not be construed as limiting the scope of the invention but merely as illustrating different examples. It should be appreciated that the scope of the disclosure includes other embodiments not discussed in detail above. Various other modifications, changes and variations which will be apparent to those skilled in the art may be made in the arrangement, operation and details of the method and apparatus disclosed herein without departing from the spirit and scope as defined in the appended claims. Therefore, the scope of the invention should be determined by the appended claims and their legal equivalents.

Alternate embodiments are implemented in computer hardware, firmware, software, and/or combinations thereof. Implementations can be implemented in a computer program product tangibly embodied in a computer-readable storage device for execution by a programmable processor; and method steps can be performed by a programmable processor executing a program of instructions to perform functions by operating on input data and generating output. Embodiments can be implemented advantageously in one or more computer programs that are executable on a programmable computer system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language. Suitable processors include, by way of example, both general and special purpose microprocessors. Generally, a processor will receive instructions and data from a read-only memory and/or a random access memory. Generally, a computer will include one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM disks. Any of the foregoing can be supplemented by, or incorporated in, ASICs (application-specific integrated circuits), FPGAs and other forms of hardware.

What is claimed is:

1. A system comprising:
a memory storing instructions; and
a processor, coupled with the memory and to execute the instructions, the instructions when executed cause the processor to implement a digital media platform for communications wherein a primary mode of communication for the digital media platform comprises audio clips posted by users, wherein other users may access and respond to such audio clips with their own audio clips.

2. The system of claim 1 wherein the digital media platform is directed to audio clips about health care.
